(19) United States
(12) Patent Application Publication
(10) Pub. No.: US 2013/0084370 A1
(43) Pub. Date: Apr. 4, 2013

(54) VISCOSITY-CONTROLLED PROCESSING OF LIQUID FOOD

(71) Applicant: KRONES AG, Neutraubling (DE)

(72) Inventor: Jörg Zacharias, Koefering (DE)

(73) Assignee: KRONES AG, Neutraubling (DE)

(21) Appl. No.: 13/630,764

(22) Filed: Sep. 28, 2012

(30) Foreign Application Priority Data
Sep. 30, 2011 (DE) 102011083881.3

(51) Int. Cl. G01N 33/14; A23L 2/46; A23L 2/02

(52) U.S. Cl. 426/231; 99/334

(57) ABSTRACT

The present invention relates to a method of controlling, by open-loop or closed-loop control, the processing of a liquid food product, comprising the steps of providing data that are obtained from mathematical modeling of the dependency of the viscosity of liquid food products on the shear rate, measuring the viscosity of the liquid food product to be processed at a predetermined shear rate at a measuring point, and controlling, by open-loop or closed-loop control, the processing of the liquid food product at a working device downstream of the measuring point in response to the viscosity measured at the measuring point and on the basis of the provided data.

### CROSS-REFERENCE TO RELATED APPLICATION

The present application claims the benefit of priority of German Application No. 102011083881.3, filed Sep. 30, 2011. The text of the priority application is incorporated herein by reference in its entirety.

### FIELD OF THE DISCLOSURE

The present disclosure relates to the open-loop or closed-loop control of the processing of a liquid food product, in particular a fruit or vegetable juice, wherein in the processing of the liquid food product, the viscosity of the latter must be taken into consideration.

### BACKGROUND DESCRIPTION OF THE RELATED ART

In the processing of liquids, the viscosity of these liquids often plays an important role. For example, viscosity must be taken into consideration in transport processes due to its influence on boundary layer processes. The functioning of process technology, such as the hydraulic transport by means of pumps, heat transfer technology, mixing processes, separation technology up to product filling, are decisively influenced by the viscosity of the liquids. The consideration of viscosity is particularly important in connection with the processing of liquid food products, for example fruit or vegetable juices, milk or beer. In the pasteurization of liquid food by flash pasteurizers, the viscosity of the liquid food product to be pasteurized must be taken into consideration in the dimensioning of the heat exchanger, for example the heating plates or heating tubes. If, due to viscosity, the area of the heat exchanger is smaller than required for successful pasteurization, the processed liquid food product is not sufficiently heated or cooled.

Viscosity, however, is a measurand that can only be obtained with difficulties during the operation of a plant for processing a liquid food product. The determination of the viscosity of non-Newtonian, in particular pseudoplastic media is particularly difficult as these media exhibit a relatively high dependency of viscosity on the shear rate, in particular at relatively low shear rates. While viscosities can be measured in the laboratory (offline), for example, by means of rotational rheometers for various shear rates, it is very difficult to obtain measured values for the viscosity of various shear rates of liquid food products occurring in operation while a plant is being operated (online).

It is known in prior art to determine online the viscosity of a liquid food product at relatively high shear rates of higher than 1000 s⁻¹. This is possible with a high reliability for Newtonian media, for example with the aid of the Promass 83 1 of the company Endress+Hauser GmbH and Co. KG. For lower shear rates and pseudoplastic media, such as in particular fruit and vegetable juices, however, no determination of viscosity, and above all no open-loop or closed-loop control of a plant for processing the media on the basis of the determined viscosity, is known. It is thus an object of the present invention to provide a method of controlling, by open-loop or closed-loop control, the processing of liquid food products that takes into consideration the viscosity of the liquid food products at a working device within a process line which normally does not lie within the range of shear rates of the measuring point or the measuring device.

### SUMMARY

The above mentioned object is achieved by the method according to claim 1. The claimed method of controlling, by open-loop or closed-loop control, the processing of a liquid food product comprises the steps of

providing data that are obtained from mathematical modeling of the dependency of the viscosity of liquid food products on the shear rate;

measuring the viscosity of the liquid food product to be processed at a predetermined shear rate at a measuring point; and

controlling, by open-loop or closed-loop control, the processing of the liquid food product at a working device downstream of the measuring point (in a process line following the measuring point) in response to the viscosity measured at the measuring point and on the basis of the provided data.

In the processing of liquid food products, among other things, the viscosity of the liquid food product to be processed at the working device must be taken into consideration to be able to ensure perfect processing. The working device can be, for example a flash pasteurizer, and the liquid food product can be a fruit or vegetable juice.

In a process line, the viscosity of the liquid food product to be processed is measured at a measuring point. However, for a perfect operation of the working device, it is not the viscosity at the measuring point that is relevant, but the viscosity at the working device itself. However, no direct measurement of the viscosity is possible here. On the other hand, the shear rate at the working device differs from that at the measuring point.

It is moreover possible for the measuring device not to measure the actual viscosity according to the present shear rate, but to perform measurements within a range of shear rates that deviates to a greater extent but is known, by its inherent measuring principle.

According to the invention, a data record is provided which represents a relationship between the viscosity and the shear rate for a number of samples of liquid food products. The data record can comprise, for example, a group of curves of viscosity versus shear rate or parameterizations of such curves. The data can moreover comprise rheological parameters that characterize individual liquid food products. The viscosity measured for the liquid food product to be processed can be related to the provided data. For example, a certain pseudoplastic model can be taken as a basis for the data and also applied to the liquid food product to be processed. For example, information on the viscosity of the liquid food product to be processed at the working device can be obtained starting from the one measured value by matching it against stored data for a liquid food product with a comparable value of viscosity at the predetermined shear rate, (also cf. detailed description below).

The temperature dependency of viscosity represents a further degree of freedom of the data record for characterizing a liquid food product.

The working device can be controlled by open-loop or closed-loop control on the basis of this data record and the viscosity of the liquid food product to be processed measured at the predetermined shear rate. Details in this respect will be described below. In any case, the method according to the invention provides an advantage in that errors in the processing of the liquid food product can be avoided since the viscosity of the liquid food product can be principally taken into consideration although it is not accessible for direct measurement at the working device. For example, the working device can be switched off if this is considered to be necessary.

According to a further development, the modeling is carried out on the basis of measurements of the viscosity of the liquid food products for at least one shear rate. So, the data are obtained on the basis of viscosity measurements and a model description of the viscosity of liquid food products in response to the shear rate. Measurements can be done in the laboratory, for example by means of a rotational rheometer. It should be emphasized that the liquid food product to be processed does not have to be identical to one of the liquid food products for which the data obtained from mathematical modeling of the dependency of the viscosity of liquid food products on the shear rate are provided, or for which modeling is carried out on the basis of measurements of the viscosity for at least one shear rate.

The modeling itself can be done on the basis of the Cox-Merz relationship. The latter in particular permits the determination of the viscosity as a function of the shear rate from oscillation measurements of viscosity (shear rate=angular frequency of the oscillation applied in the oscillation measurement). According to the Cox-Merz relationship, the shear viscosity is well in accordance with the absolute value of the complex viscosity at the same shear rate and angular frequency.

In general, pseudoplastic liquid food can be described by quite a high number of mathematical models with and without yield point. These are also referred to as viscosity function or flow curve. The flow curves with the best correlation for juices are those according to Casson, Bingham, Herschel-Bulkley, or Ostwald-de-Waele.

In particular, the modeling can be done according to the Ostwald-de-Waele relationship η=K\dot{γ}^{m-1}, wherein η and \dot{γ} designate viscosity and shear rate, and K and m designate consistency and flow index. In this case, the data on the basis of which the open-loop or closed-loop control of the processing of the liquid food product is done at the working device can comprise values for m (and possibly K) for a multiplicity of liquid food products, wherein the method comprises determining the value of m for the liquid food product and controlling, by open-loop or closed-loop control, is done on the basis of the determined value of m (and possibly K) for the liquid food product (see detailed description below). The Ostwald-de-Waele relationship is particularly suited for the description of the relationship of viscosity and shear rate of flowing fruit or vegetable juices.

In particular, recipes can be provided which contain information on marginal products, e. g., values for m and K. The data values for the marginal products can span a parameter space defining parameter ranges for which perfect processing is possible.

According to a further development, the processing of the liquid food product at the working device can be stopped when the determined value for m for the liquid food product exceeds a first predetermined limit and/or falls below a second predetermined limit. In this manner, it can in particular be prevented that rejects are produced or repeated processing becomes necessary. The provided data can comprise data records for recipes for various types of liquid food products. Each data record here comprises upper and lower limits corresponding to marginal products. The first and second predetermined limits can be read out of one of the data records.

In general, in the above-described examples of the method according to the invention, in response to the measurement of the viscosity of the liquid food product, a flow rate of the liquid food product can be increased or reduced, and/or a pressure of the flow rate of the liquid food product can be increased or reduced. In addition or as an alternative, the liquid food product can be, in response to the measurement of the viscosity of the liquid food product, diluted or thickened. For example, on the basis of the viscosity measurement and the provided data, it can be determined that the viscosity of a fruit juice (as liquid food product) to be pasteurized at the flash pasteurizer (as working device) is so low that sufficient heating and optionally subsequent cooling is not ensured by the dimensions of the plates or the tube of the flash pasteurizer. The required temperature profile is not reached in this case. In this case, the juice can be thickened in response to this (increased fruit proportion), or the flow rate or pressure can be reduced. Equally, one can react online to pressure losses, for example by adapting/controlling by open-loop/closed-loop control the flow rate of the liquid food product to be processed.

As will be clear from the above illustration, in the present application, the consideration of the dependency of the viscosity of liquid food products on the shear rate plays an important role. The viscosity of the liquid food product to be processed is measured at a predetermined shear rate. According to an exemplary Ostwald-de-Waele relationship, for example, one can draw conclusions from this measurement on the viscosity at a shear rate different from the predetermined one. The locally present mean shear rate of a liquid food product can be, for example, according to the approximation formula for pipeline transport

$${γ}^{\overline{.}} = \frac{4 \dot{V}}{π {R}^{3}}$$

It in particular describes the shear rate at the tube wall decisive for heat transfer. So, the mean shear rate {dot over ( γ̅ calculated with this formula is a function of the flow rate \dot{V} and the present geometry of the pipeline system as it is stated by the radius R of the tube.

The above mentioned object is also achieved by an open-loop or closed-loop control device for a plant for processing a liquid food product, comprising

a memory for storing data that are obtained from mathematical modeling of the dependency of the viscosity of liquid food products on the shear rate (e. g. as described above in connection with the method according to the invention); and

a control unit for controlling, by open-loop or closed-loop control, the processing of the liquid food product at a working device downstream of a measuring point in response to a viscosity of the food product to be processed measured at the measuring point at a predetermined shear rate and on the basis of the stored data.

Equally, the above mentioned object is achieved by a processing device, in particular a flash pasteurizer, for the processing of a liquid Newtonian or pseudoplastic food product, in particular a fruit or vegetable juice, comprising

a measuring point for measuring the viscosity of the liquid food product at a predetermined shear rate of the liquid food product;

a working device for processing the liquid food product, the working device being provided downstream of the measuring point; and

the above mentioned open-loop or closed-loop control device.

All embodiments of the above-described method can be implemented in the mentioned device.

In all above mentioned examples, the liquid food product can also be a (lumpy) suspension or pulp or mash or a liquid containing fibers. It can also be one of liquid food products with defined flow limits Suspensions that tend to wall-slip effects can also be included here.

According to the above description, it is principally possible to measure the viscosity of a liquid food product to be processed at the measuring point at a predetermined shear rate and to then draw conclusions about the viscosity at the working device knowing the shear rate at this point. Thus, a method of determining the viscosity of a liquid food product at a working device for processing the liquid food product is furthermore also provided, comprising the steps of

providing data that are obtained from mathematical modeling of the dependency of the viscosity of liquid food products on the shear rate (for example on the basis of the Ostwald-de-Waele relationship);

measuring the viscosity of the liquid food product at a predetermined shear rate of the liquid food product at a measuring point; and

determining the viscosity of the liquid food product at a second shear rate of the liquid food product differing from the predetermined shear rate on the basis of the stored data.

The working device can be controlled in response to the viscosity determined in this manner

Below, embodiments of a method according to the present disclosure will be described with reference to the drawing. The described embodiments are to be considered in any respect only as illustrative and not as restrictive, and various combinations of the stated features are included in the present disclosure.

### BRIEF DESCRIPTION OF THE DRAWINGS

FIG. 1 illustrates a process line with a measuring device for measuring the viscosity of a liquid food, a working device for processing the liquid food, and a control device for controlling the working device.

FIG. 2 shows, by way of example, measuring results for the viscosity of a xanthane sample in response to the shear rate in a double logarithmic representation, and a line of best fit for the confirmation of the Ostwald-de-Waele relationship.

FIG. 3 shows, for exemplary marginal products, the Ostwald-de-Waele relationship for corresponding flow indices mₜₒₚ and m_{bottom} of the marginal products.

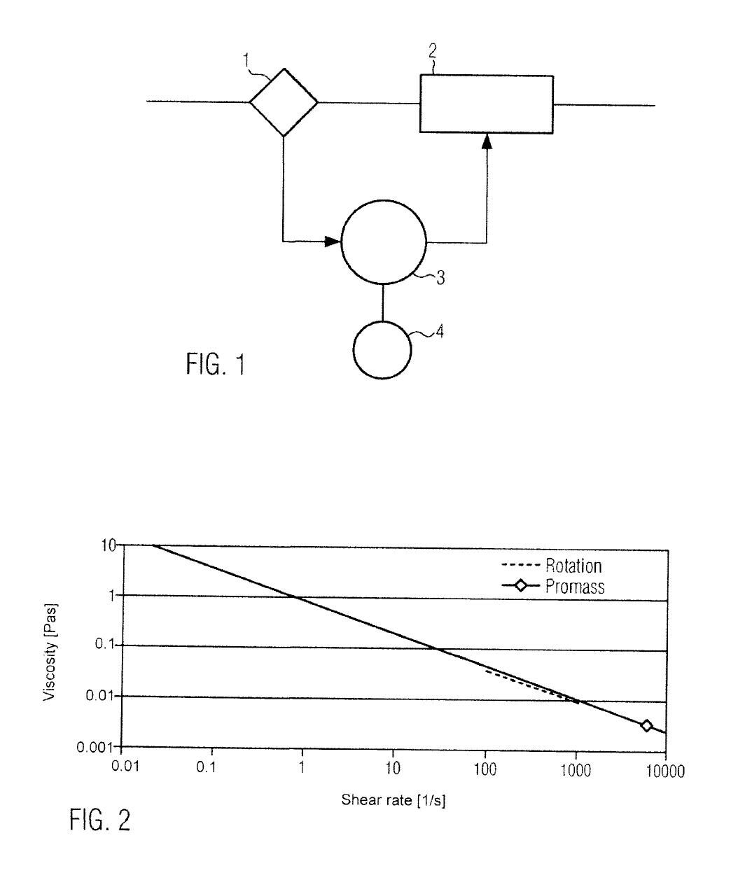

FIG. 1 illustrates a process line with a measuring device 1 for measuring the viscosity of a liquid food, a working device 2 for processing the liquid food, and a control device 3 for controlling the working device based on the one hand on the measured viscosity value and on the other hand on data that are stored in a memory 4. In the shown embodiment, the liquid food can be a fruit juice, and the working device 2 can be a flash pasteurizer or a UHT pasteurizer used for the pasteurization of the fruit juice. Below, it will be furthermore assumed that the working device 2 is a flash pasteurizer (FP) used for heating fruit juice. Of course, the invention is not restricted to a working device 2 or to a liquid food product specified in this manner

### DETAILED DESCRIPTION OF THE PREFERRED EMBODIMENTS

The dimensioning of the FP principally depends on the viscosity of the liquid food to be treated. When it is supplied to a customer, the FP, that can be a plate heat exchanger or a tubular heat exchanger, is dimensioned for typical applications. However, it cannot be excluded that the customer operates a process line with the FP for juices whose viscosity does not permit sufficient heating by the FP. Among other things, the present invention serves to avoid the production of rejects in such a case.

The viscosity of the juice to be pasteurized is measured at the measuring device 1 at a predetermined shear rate. Such measurement can be done, for example, with the aid of the Promass 83 1 of the company Endress+Hauser GmbH and Co. KG. For example, the predetermined shear rate at which viscosity is measured can be 5000 s⁻¹. The measured viscosity is entered into the control device 3. The latter can access the memory 4 which stores data on a plurality of liquid food products which can differ from the considered liquid food product to be processed (in particular as to its pseudoplastic property). The data are based on modeling of the liquid food products describing a relationship between their respective viscosities and shear rates. So, in the present example, the data are acquired for a number of fruit juices and stored in the memory 4 before the process line is commissioned.

In particular, recipes for various juices or types of juices can be stored in the memory 4 which each characterize a plurality of liquid food products, where they comprise values for marginal products such that these values define a parameter range within which perfect processing at the working device 2 is permitted.

More precisely, in this example, the Ostwald-de-Waele relationship η=K\dot{γ}^{m-1} is applied, wherein η and \dot{γ} designate viscosity and shear rate, and K and m designate consistency and flow index. K and m are determined for a number of fruit juices by rotational rheometry. For this, measurements at least of the viscosity at several shear rates are carried out for each juice sample, from which then the rheological parameters K and m can be determined, assuming the validity of the Ostwald-de-Waele relationship, the parameters decisively determining the pseudoplastic properties of the juices.

FIG. 2 shows an example of measurements in a double logarithmic representation and the resulting line of best fit, taking a xanthane sample as example. Xanthane is a common ingredient of beverages and exhibits similar flow properties as many juices. Measured values are shown which have been obtained from rotational rheometrical measurements for shear rates within the range relevant for process technology of ca. 100 to ca. 1000 s⁻¹, and a measured value of a measurement with the aid of the Promass 83 1 (Promass measurement) performed at a shear rate of 5000 s⁻¹ is shown. The flow index m can be clearly determined from the slope of the line of best fit. K and m are stored for all juice samples for which the FP can guarantee sufficient pasteurization.

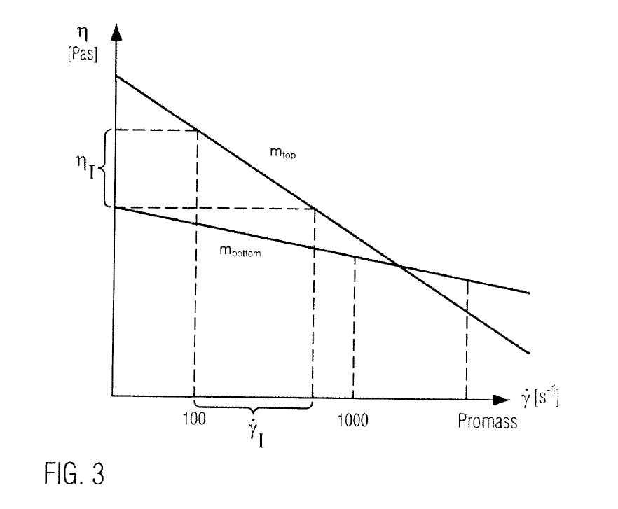

FIG. 3 by way of example shows (not to scale) the dependency of the viscosity on the shear rate for experimentally determined marginal products of a selected recipe for which the FP still barely operates reliably. A maximum slope mₜₒₚ and a minimum slope m_{bottom} result which correspond to a viscosity interval \dot{γ}₁ in which viscosities occur at the working device 4 for shear rates that realistically occur in the interval \dot{γ}₁ that depend on the design of the working device 4 and typically applied flow rates, for which viscosities perfect processing can be guaranteed. If there are viscosities above the interval \dot{γ}₁, overheating occurs. For viscosities below the interval \dot{γ}₁ , heating is not sufficient. If now based on the measurement of the viscosity of the liquid food product to be processed, a value for m is determined which is above mₜₒₚ or below m_{bottom}, the process line, in particular the pasteurization, at the FP can be stopped. As an alternative, the flow rate or the pressure or the dilution/thickening of the liquid food product can be controlled by open-loop or closed-loop control, so that the viscosity of the liquid food product to be processed changes at the FP until it can work perfectly. An analogous procedure can be applied in the case of a cooling of the liquid food product.

In the simplest case, an operator of the process line could enter K and m of a liquid food product to be processed into the control device 3 which can then determine, by directly matching the stored m values, whether the liquid food product to be processed is suited for pasteurization with the aid of the FPs. In general, the operator will not have any knowledge about the exact rheological parameters. So, viscosity is measured by a Promass measurement with the aid of the measuring device 1. By matching against the data stored in the memory 4, the control device 3 can decide, in particular after a preselection of a recipe, whether the respective liquid food product, here the fruit juice, is suited for processing by the working device 2, here the FP. One can determine, for example, which stored viscosity value for the predetermined shear rate at which the measuring device 1 performs the viscosity measurement, that means here e.g. 5000 s⁻¹, comes closest to the viscosity value measured by the measuring device 1. If the corresponding m value of the straight line matching this viscosity value is within the viscosity-shear rate diagram between m_{bottom} and mₜₒₚ, the control device 3 will decide that processing by the working device 2 can be successfully done.

In one variant, the described system can be combined with a second or third system determining other parameters, such as color or density or conductivity or pH value, etc., to ensure and improve the unambiguousness and reliability of the viscosity measurement (quasi by a cross-correlation with redundant data).

An operator of the system must select the correct recipe. If he selects, intentionally or unintentionally, a wrong recipe for another product, naturally, no perfect processing of the liquid food product is guaranteed.

It is assumed that the process line comprises a Promass 83 1 as measuring device 1. Of course, another device can be used. Measurements with the Promass 83 1 can be calibrated by extrapolating Promass measuring points for a shear rate of 5000 s⁻¹ according to the Ostwald-de-Waele relationship with a known m and K to a comparison viscosity, for example 500 s⁻¹, and comparing them with a rotational rheological comparison measurement. The deviation can be used for calibration (shifting of the straight line in the double logarithmic representation). It can also be advantageous to correct the Promass measurement by a system-related wall-slip rate. Moreover, a temperature correction, for example for considering the decrease in dynamic viscosity as temperature rises, can be applied according to the Arrhenius-Andrade relationship or the Vogel-Fulcher-Tammann equation. Moreover, where suspensions are to be processed, a correction of the measured viscosity according to the Einstein model, η=η₀(1+2.5Φ) can be effected, wherein no is the viscosity of the suspension liquid and Φ<<1 is the volume fraction of solids.

What is claimed is:

1. Method of controlling, by open-loop or closed-loop control, the processing of a liquid food product, comprising the steps of
providing data that are obtained from mathematical modeling of the dependency of the viscosity of liquid food products on the shear rate;
measuring the viscosity of the liquid food product to be processed at a predetermined shear rate at a measuring point; and
controlling, by open-loop or closed-loop control, the processing of the liquid food product at a working device downstream of the measuring point in response to the viscosity measured at the measuring point and on the basis of the data.

2. Method according to claim 1, wherein the modeling is effected on the basis of measurements of the viscosity of the liquid food products for at least one shear rate.

3. Method according to claim 1, wherein the modeling is effected based on the Cox-Merz relationship.

4. Method according to claim 1, wherein the modeling is effected according to the Ostwald-de-Waele relationship η=K\dot{γ}^{m-1}, wherein Ti and y designate the viscosity and the shear rate, and K and m designate the consistency and the flow index.

5. Method according to claim 4, wherein the data comprise values for m for a plurality of liquid food products, the method comprising the determination of the value of m for the liquid food product, and controlling, by open-loop or closed-loop control, is done on the basis of the determined value of m for the liquid food product.

6. Method according to claim 5, wherein the value of m for the liquid food product to be processed is entered by a user into a device for controlling, by open-loop or closed-loop control, the processing of the liquid food product.

7. Method according to claim 5, wherein the processing of the liquid food product at the working device is stopped when the determined value for m for the liquid food product exceeds a first predetermined limit and/or falls below a second predetermined limit.

8. Method according to claim 7, wherein the provided data comprise data records for recipes for various types of liquid food products, and the first and the second predetermined limits are read out from one of the data records.

9. Method according to claim 1, wherein in response to the measurement of the viscosity of the liquid food product, a flow rate of the liquid food product is increased or reduced, and/or a pressure of the flow rate of the liquid food product is increased or reduced, and/or the liquid food product is diluted or thickened.

10. Method according to claim 1, wherein the working device is a flash pasteurizer.

11. Method according to claim 1, wherein the liquid food product is one of a fruit juice, a fruit suspension, a vegetable juice or a vegetable suspension.

12. Method according to claim 1, further comprising determining the shear rate of the liquid food product to be processed at the working device on the basis of the formula
$${γ}^{\overline{.}} = \frac{4 \dot{V}}{π {R}^{3 }} ,$$
wherein the mean shear rate is designated with {dot over ( γ̅, and the flow rate of the liquid food is designated with \dot{V}, and the radius of a tube through which the liquid food flows is designated with R, and wherein
the controlling, by open-loop or closed-loop control, of the processing of the liquid food product at the working device is effected on the basis of the shear rate determined in this manner.

13. Method according to claim 1, wherein the measured viscosity of the liquid food product to be processed undergoes a temperature correction, and/or the data that are obtained from mathematical modeling of the dependency of the viscosity of liquid food products on the shear rate contain a temperature correction.

14. Method according to claim 1, wherein the liquid food product is present as a suspension and the measured viscosity of the liquid food product to be processed undergoes a correction according to the volume fraction of solids of the suspension, and/or the data that are obtained from mathematical modeling of the dependency of the viscosity of liquid food products on the shear rate contain a correction according to the volume fraction of solids of the suspension, wherein the correction is effected in particular according to the Einstein model.

15. Open-loop or closed-loop control device for a plant for processing a liquid food product, comprising:
a memory for storing data that are obtained from mathematical modeling of the dependency of the viscosity of liquid food products on the shear rate; and
a control unit for controlling, by one of open-loop or closed-loop control, the processing of the liquid food product at a working device downstream of a measuring point in response to a viscosity of the food product to be processed measured at the measuring point at a predetermined shear rate and on the basis of the stored data.

16. Processing device for processing a liquid food product, comprising:
a measuring point for measuring the viscosity of the liquid food product at a predetermined shear rate of the liquid food product;
a working device for processing the liquid food product, wherein the working device is provided downstream of the measuring point; and
the open-loop or closed-loop control device according to claim 15.

17. Processing device according to claim 16, wherein the working device is a flash pasteurizer for flash pasteurization of a fruit juice, a fruit suspension, a vegetable juice, or a vegetable suspension as the liquid food product.

18. Method of determining the viscosity of a liquid food product at a working device for processing the liquid food product, comprising:
providing data that are obtained from mathematical modeling of the dependency of the viscosity of liquid food products on the shear rate;
measuring the viscosity of the liquid food product at a predetermined shear rate of the liquid food product at a measuring point; and
determining the viscosity of the liquid food product at a second shear rate of the liquid food product differing from the predetermined shear rate on the basis of the stored data.
